(19) United States
(12) Patent Application Publication
(10) Pub. No.: US 2012/0172851 A1
(43) Pub. Date: Jul. 5, 2012

(54) ND:YAG LASER APPARATUS

(75) Inventors: Hee Chul Lee, Gyeonggi-Do (KR); Hae Lyung Hwang, Gyeonggi-Do (KR)

(73) Assignee: LUTRONIC CORPORATION, Goyang-Si, Gyeonggi-Do (KR)

(21) Appl. No.: 13/382,645

(22) PCT Filed: Jul. 7, 2009
(86) PCT No.: PCT/KR2009/003694

§ 371 Date: Jan. 26, 2012

(51) Int. Cl. A61B 18/24

(52) U.S. Cl. 606/3

(57) ABSTRACT

Disclosed herein is a fat removal 1414 nm Nd:YAG laser apparatus for direct irradiation into fat. The apparatus includes a flashlamp which is supplied the electrical power from the power supply unit and emitting light; an Nd:YAG rod radiate a laser wavelength and amplify a laser wavelength after absorbed the pumping light from flashlamp; a high reflector and an output coupler located on two sides of the Nd:YAG rod and configured to reflect laser wavelength radiated from the Nd:YAG rod; a convergence lens for converging a laser beam; an optical fiber for delivery the laser beam; and a cannula coupled to the output end of the optical fiber and configured to allow the optical fiber to extend underneath skin without being bent, thereby delivery the laser beam to hypodermic fat. Both end surfaces of the Nd:YAG rod, the internal surface of the high reflector, and the internal and external surfaces of the output coupler are coated in order to obtain only the laser beam with the wavelength of 1414 nm wavelength.

### TECHNICAL FIELD

The present invention relates, in general, to an Nd:YAG laser apparatus, and, more particularly, to a fat removal 1414 nm wavelength Nd:YAG laser apparatus for direct irradiation into fat, which is capable of directly irradiating a laser beam into hypodermic fat rather than irradiating a laser beam from outside the skin, and which is capable of efficiently removing fat and minimizing side effects on adjacent tissues by using a laser beam with a wavelength of 1414 nm, which can be obtained by the Nd:YAG laser apparatus and has high absorption coefficients for both fat and water.

### BACKGROUND ART

The wavelengths of fat removal lasers being currently commercially sold include 1064 nm, 1319 nm and 1444 nm.

FIG. 1 is a graph showing the absorption coefficients for fat and water at various wavelengths (the graph shown in FIG. 1 is cited from U.S. Pat. No. 6,605,080).

From the graph of FIG. 1, it can be seen that the 1414 nm wavelength has much higher absorption coefficients for water and fat than the 1064 nm, 1319 nm, 1338 nm and 1357 nm wavelengths and has a slightly higher absorption coefficient for fat and a slightly lower absorption coefficient for water than the 1444 nm wavelength.

Furthermore, the 1064 nm, 1319 nm, 1338 nm, 1357 nm, 1414 nm and 1444 nm wavelengths are all wavelengths that can be obtained by the Nd:YAG laser.

Meanwhile, according to a prior art method for removing fat using an Nd:YAG laser, fat is removed using 1064 nm and 1319 nm laser beams that have low absorption coefficients for fat and water.

FIG. 2 shows experimental results that were measured using Optical Coherence Tomography (OCT) after 1064 nm, 1319 nm and 1414 nm laser beams were irradiated into a pig's fat, which is similar to human fat.

From the results of FIG. 2, it can be seen that the 1414 nm wavelength is superior to other two wavelengths from the point of view of the ablation efficiency.

A fat cell is composed of 60˜85% lipids, 5˜30% water and 2˜3% protein.

Accordingly, the prior art method for removing fat using an Nd:YAG laser has the following problems.

That is, the prior art method for removing fat using an Nd:YAG laser that radiates a wavelength, such as the 1064 nm or 1319 nm wavelength, that has lower absorption coefficients for water and fat than the 1414 nm wavelength has a problem in that a laser beam propagate to tissues adjacent to the fat during the removal of the fat, as shown in FIG. 3, because the 1064 nm and 1319 nm laser beam have a low absorption coefficient for the fat.

In the case where the 1064 nm and 1319 nm laser beam propagate to tissues adjacent to the fat as described above, these wavelength harms human tissues because it has also a low absorption coefficient by water.

That is, when the prior art method using a 1064 nm and 1319 laser beam are used, fat cannot be effectively removed and adjacent tissues are harmed by using these wavelengths.

Furthermore, although a laser beam with a wavelength around the 1200 nm wavelength which has a high absorption coefficient for fat is used, the laser beam has a low absorption coefficient for water as shown in FIG. 3, so that adjacent tissues can be harmed in the case where a user erroneously irradiates the laser beam into the adjacent tissues, with the result that a problem occurs also in this case.

### DISCLOSURE OF INVENTION

### Technical Problem

Accordingly, the present invention has been made keeping in mind the above problems occurring in the prior art, and an object of the present invention is to provide a fat removal 1414 nm Nd:YAG laser apparatus for direct irradiation into fat, which is adapted such that an optical fiber and a cannula are directly inserted into fat underneath skin, so that a laser beam can be considering only the absorption coefficient for fat without considering the loss of energy resulting from the absorption by water.

Another object of the present invention is to provide a fat removal 1414 nm Nd:YAG laser apparatus for direct irradiation into fat, this laser beam has high absorption coefficients for both fat and water, so that fat can be removed efficiently and, at the same time, damage to adjacent tissues can be minimized.

Still another object of the present invention is to provide a fat removal 1414 nm Nd:YAG laser apparatus for direct irradiation into fat, which is adapted such that an optical fiber is separated into a fixed optical fiber configured to deliver a laser beam and a disposable optical fiber configured to be actually inserted into a human body and irradiate the laser beam into the fat, so that the cost burden resulting from the disposal of an optical fiber can be reduced in the case where the optical fiber used in treatment is disposed of and the convenience of use can be improved by eliminating the sterilize of the optical fiber whenever treatment using the laser apparatus is performed.

### Technical Solution

In order to accomplish the above objects, the present invention provides a fat removal 1414 nm Nd:YAG laser apparatus for direct irradiation into fat, including a flashlamp that is supplied the electrical power from a power supply unit and emitting light; an Nd:YAG rod which radiate the laser wavelength after absorbing the light from the flashlamp; a high reflector and an output coupler located on two sides of the Nd:YAG rod and configured to reflect light output from the Nd:YAG rod; a convergence lens for converging a laser beam radiate from the output coupler; an optical fiber for delivery the laser beam converged by the convergence lens; and a cannula coupled to the output end of the optical fiber and configured to allow the optical fiber to extend underneath skin without being bent, thereby delivery the laser beam by the optical fiber to hypodermic fat; wherein both end surfaces of the Nd:YAG rod, the internal surface of the high reflector, and the internal and external surfaces of the output coupler are coated in order to obtain the only a laser beam with a wavelength of 1414 nm wavelength.

### Advantageous Effects

The above-described fat removal 1414 nm Nd:YAG laser apparatus for direct irradiation into fat according to the present invention has the following effects.

First, since the apparatus of the present invention is configured to oscillate only a 1414 nm laser beam and can use the 1414 nm laser beam to remove fat as described above, a laser beam having the highest absorption coefficient for fat, selected from various wavelengths that can be obtained by an Nd:YAG laser, can be used, with the result that there is an advantageous effect of removing fat very effectively.

Second, when a 1414 nm laser beam is used as described above, there may occur a case where the 1414 nm laser beam is not entirely absorbed by fat F but is propagated to adjacent tissues, in which case the 1414 nm wavelength has a absorption coefficient for water because a fat cell contains a considerable amount of water, with the result that there is an excellent effect of reducing damage to adjacent tissues. In practice, when calculation is performed with the scattering coefficient disregarded and the absorption coefficient being considered, 99% of the 1414 nm wavelength is absorbed when it is propagated 2 mm because it has a high absorption coefficient for water, while the 1064 nm wavelength must be propagated 31 cm until 99% of the 1064 nm wavelength must be absorbed.

Third, since an optical fiber for delivery a laser beam is separated into a fixed optical fiber and a disposable optical fiber actually inserted into a human body and configured to irradiate a laser beam into the human body, it is necessary to dispose of only the disposable optical fiber 172 inserted into the human body, so that there is an advantage of reducing the cost burden resulting from the disposal of an optical fiber in the case where the optical fiber used in treatment is disposed of, and there is a further advantage of improving the convenience of use by eliminating sterilize of the optical fiber whenever treatment using the laser apparatus is performed.

Moreover, there is the excellent effect of reducing damage to adjacent tissues because the absorption coefficient of the 1414 nm wavelength for water is very high.

### BRIEF DESCRIPTION OF DRAWINGS

The above and other objects, features and advantages of the present invention will be more clearly understood from the following detailed description taken in conjunction with the accompanying drawings, in which:

FIG. 1 is a graph showing the absorption coefficients of fat and water;

FIG. 2 presents photos showing experimental results that were measured using Optical Coherence Tomography (OCT) after 1064 nm, 1319 nm and 1414 nm laser beams were irradiated into a pig's fat;

FIG. 3 is a conceptual diagram showing the case where a 1064 nm laser beam is used according to the prior art;

FIG. 4 is a conceptual diagram showing the case where a laser beam with a wavelength around 1200 nm is used;

FIG. 5 is a graph plotting reflectivities that enable laser beams with other wavelengths to be simultaneously oscillated when the reflectivity of an output coupler at the 1414 nm wavelength is 93%;

FIG. 6 is a graph plotting the reflectivity curve of output couplers that enables laser beams with other wavelengths to be simultaneously oscillated when the reflectivity of an output coupler at the 1414 nm wavelength is 93%;

FIG. 7 is an energy level diagram showing the energy levels of an Nd:YAG crystal at room temperature and at a temperature higher than room temperature;

FIG. 8 is a graph plotting output energy against the temperature of cooling water in the case where input energy is 44 J;

FIG. 9 is a graph plotting the output energy of the 1414 nm wavelength and the 1444 nm wavelength against the temperature of cooling water;

FIG. 10 is a diagram showing the construction of the fat removal 1414 nm Nd:YAG laser apparatus for direct irradiation into fat according to the present invention;

FIG. 11 is a detailed sectional view of the cannula shown in FIG. 10;

FIG. 12 is a diagram showing an example of the use of the cannula of FIG. 11 with a separated disposable optical fiber which has a straight type.

FIG. 13 is a diagram showing an example of the use of the cannula of FIG. 11 with a separated disposable optical fiber which has a curved shape.

FIG. 14 is a conceptual diagram showing the case where a 1414 nm wavelength laser beam is used according to the present invention.

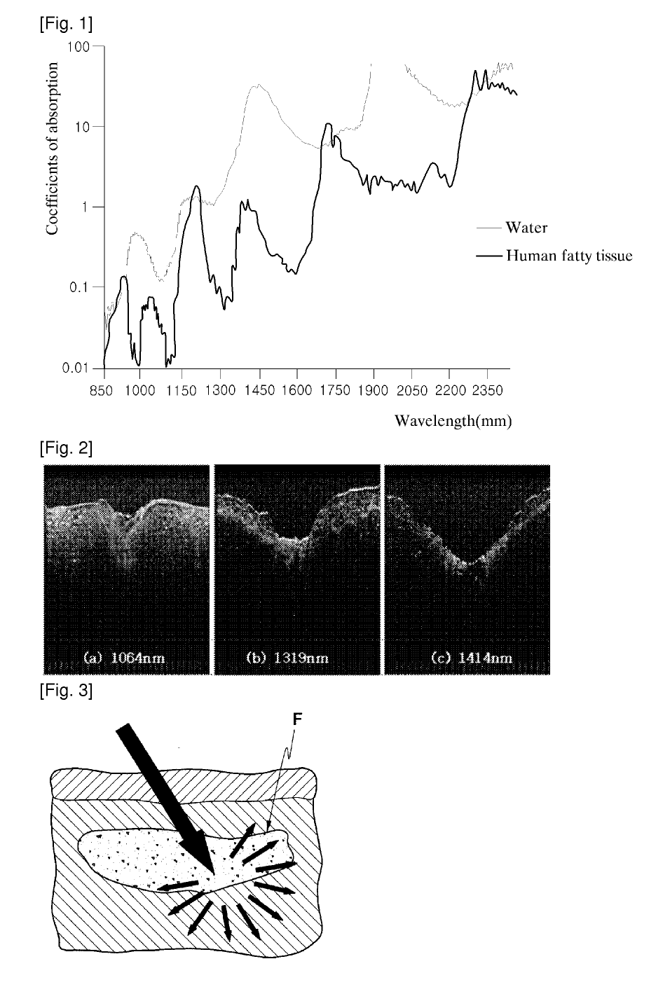

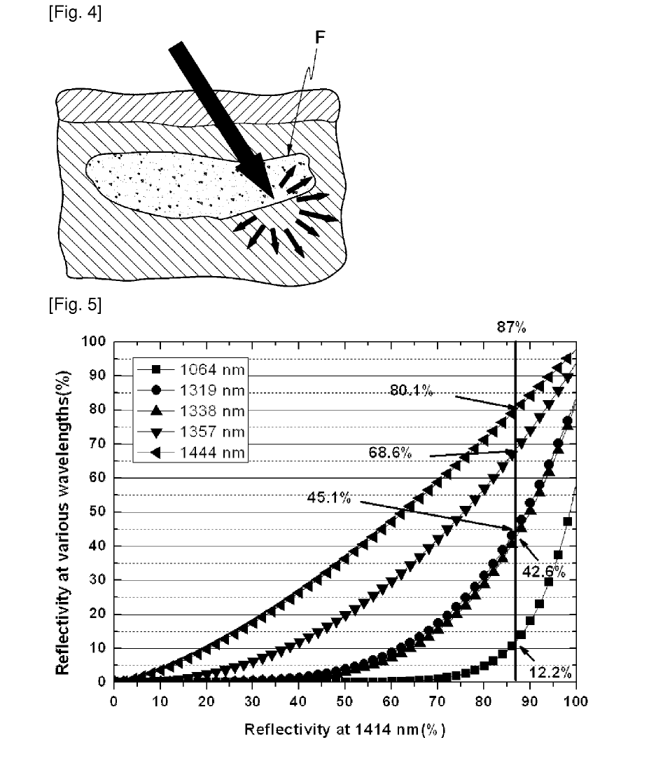

### MODE FOR THE INVENTION

A preferred embodiment of a fat removal 1414 nm Nd:YAG laser apparatus for direct irradiation into fat according to the present invention will be described in detail below with reference to the accompanying drawings.

First, a method of obtaining a 1414 nm laser beam will be schematically described below with reference to the following publications.
[1] Hee Chul Lee, “Simultaneous dual-wavelength oscillation at 1357 nm and 1444 nm in a Kr-flashlamp pumped Nd:YAG laser”, Opt Comm. Vol. 281 2008 pp. 4455˜4458.
[2] Richard C. Powell, Physics of solid-state laser materials, Springer-Verlag, 1998, Chap. 8.
[3] W. Koechner, Solid-State Laser Engineering, Springer-Verlag, 1999, pp. 48.

Since the 1414 nm and 1444 nm wavelengths have smaller stimulated emission cross sections than the 1064 nm or 1319 nm wavelength, it is very difficult to obtain them. Hitherto it has been known that the stimulated emission cross section and branching ratio of the 1444 nm wavelength are greater than those of the 1414 nm wavelength. However, the 1414 nm and 1444 nm wavelengths do not have the large difference in stimulated emission cross section and branching ratio, so that there is a strong possibility of the two wavelengths being oscillated at the same time.

The present invention is intended to demonstrate that with regard to an Nd:YAG crystal, the 1414 nm wavelength is superior than 1444 nm wavelength due to the energy level characteristics of the two wavelengths in actual laser operation in Nd:YAG laser, and is also intended to implement the fat removal Nd:YAG laser apparatus using the 1414 nm wavelength.

1414 nm wavelength is difficult to obtain in Nd:YAG laser because it has a small stimulated emission cross section. However, when a method of simultaneous dual-wavelength oscillation in a one laser crystal is used, it is possible to find the required reflectivity of output coupler for single oscillation of 1414 nm wavelength.

In an example (method 1), the reflectivities for simultaneous dual-wavelength oscillation at the 1064 nm and 1319 nm wavelengths can be calculated using the following Equation 1 (the reflectivities at the high reflector are the same) (Publication [1]).

$$ln \frac{1}{{r}_{2}} = 2 α L ( \frac{{σ}_{2} {v}_{1}}{{σ}_{1} {v}_{2}} - 1 ) + \frac{{σ}_{2} {v}_{1}}{{σ}_{1} {v}_{2}} ln ( \frac{1}{{r}_{1}} ) \qquad ( 1 )$$

Here,

σ, ν

and

r
are a stimulated emission cross section, a laser frequency and reflectivity at an output coupler, respectively. The subscripts 1 and 2 denote the 1064 nm wavelength and the 1319 nm wavelength, respectively.

### L

and
α
are the length of the Nd:YAG crystal and the loss coefficient, respectively.

For example, if, as a result of the calculation using Equation 1, the required reflectivity of Output coupler for simultaneous dual-wavelength oscillation at 1064 nm and 1414 nm were 25% and 93%, respectively. It means that if the output coupler has the reflectivity of less than 26% at 1064 nm wavelength then only the 1414 nm wavelength can be obtained.

Accordingly, when the above-described process is applied to the other possible lasing wavelengths that can be obtained by a Nd:YAG crystal, the 1414 nm or 1444 nm wavelength which has a comparatively very small stimulated emission cross section can be obtained when the reflectivities of the output coupler is less than a specific reflectivities at other possible lasing wavelengths.

That is, when, for example, the reflectivity of the output coupler for the 1414 nm wavelength is 93%, as shown in FIG. 5, the reflectivities for the 1064 nm, 1319 nm, 1338 nm, 1357 nm and 1444 nm wavelengths must be less than 25%, 60%, 58%, 79% and 88%, respectively, in order to obtain only the 1414 nm wavelength.

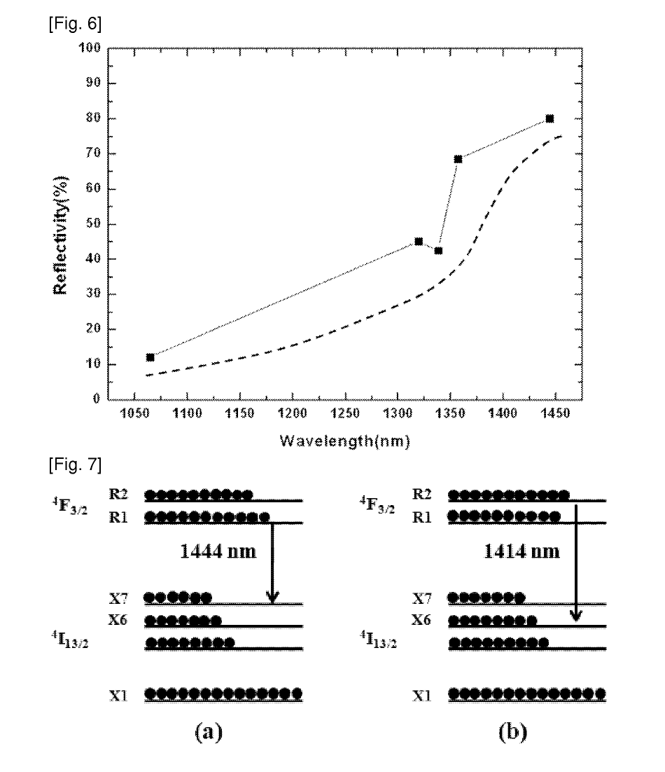

Also, as shown in FIG. 6, when the output coupler has the reflectivities below than dotted line, only the 1414 nm wavelength can be obtained.

Another method of obtaining only the 1414 nm wavelength (method 2) is to use a laser line filter as an output coupler, that is, which has the appropriate reflectivity for the 1414 nm wavelength and very low reflectivities for all other possible lasing wavelengths, thereby preventing the lasing of the wavelengths without 1414 nm wavelength.

Although it is possible to actually manufacture such minors, it is generally very difficult to manufacture such minors so that they can have reflectivities equal to or greater than 99%, in general it has a low damage threshold.

Still another method (method 3) is to cause loss to the other possible lasing wavelengths at the high reflector and the output coupler.

That is, in method 1, the reflectivity of the high reflector has a reflectivity of 100% for all the possible lasing wavelengths.

The prevention of generation by setting reflectivities to values less than 25%, 60%, 58%, 79% and 88% for wavelengths of 1064 nm, 1319 nm, 1338 nm, 1357 nm and 1444 nm means the prevention of oscillation by increasing the loss of respective wavelengths inside the resonator. Therefore, 1414 nm can obtain by increase the loss of the other possible lasing wavelengths at the high reflector and output coupler.

Another method of obtaining only the 1414 nm wavelength (method 4) is to use a saturable absorber or selective absorber. Saturable absorber has the two types of liquid and crystal and selective absorber which consist of semi conductor array. Due to They can absorb the special spectral range, it can be use the optical component for increase the losses in the resonator at the possible lasing wavelengths without 1414 nm wavelength.

Accordingly, the causing of the loss of quantities corresponding to the above-described numerical values at the output coupler and the high reflector can also be a method of acquiring only the 1414 nm wavelength.

TABLE 1
Stimulated emission cross sections at wavelengths of 1414 nm and
1444 nm in Nd: YAG crystal (Publication 2)

| | | Stimulated | |
|---|---|---|---|
| | Wavelength | emission cross | Branching |
| Transition | (nm) | section (10⁻²⁰cm²) | ratio |
| ⁴F_{3/2} →⁴I_{11/2} | 1064.2 | 30 | 0.1275 |
| (R₂-Y₃) | | | |
| ⁴F_{3/2} →⁴I_{13/2} | 1318.7 | 9.5 | 0.0183 |
| (R₂-X₁) | | | |
| ⁴F_{3/2} →⁴I_{13/2} | 1338.1 | 10 | 0.0243 |
| (R₂-X₃) | | | |
| ⁴F_{3/2} →⁴I_{13/2} | 1357.2 | 7.3 | 0.0214 |
| (R₁-X₄) | | | |
| ⁴F_{3/2} →⁴I_{13/2} | 1414 | 2.0 | 0.0099 |
| (R₂-X₆) | | | |
| ⁴F_{3/2} →⁴I_{13/2} | 1444.4 | 2.8 | 0.0128 |
| (R₁-X₇) | | | |

FIG. 7 shows energy levels at which the 1414 nm and 1444 nm wavelengths are obtained in an Nd:YAG crystal.

According to Boltzmann distribution, at room temperature, only 40% of the ⁴F_{3/2}-level electron density is present in R₂ and the remaining 60% density is present in R₁ (the reason for this is that the energy of R₂ is 11509 cm⁻¹ and the energy of R₁ is 11425 cm⁻¹ (Publication [3])).

As shown in Table 1 and FIG. 7(a), the stimulated emission cross section for the 1444 nm wavelength is higher than that for the 1414 nm wavelength, so that the 1444 nm wavelength should be oscillated at room temperature. In contrast, as shown in FIG. 7(b), as increased temperature according to Boltzmann distribution, the densities of upper-levels in energy levels are increased.

Furthermore, once oscillation is performed by ions at the R₂ level, electrons are supplied from R₁ due to thermal transition (Publication [3]).

Accordingly, if the difference in reflectivity between the two wavelengths is minimal, the lasing wavelength shifts from 1444 nm to 1414 nm when the temperature of the laser crystal increases. Lasing takes place by ions at the R₂ level, electrons are replenished from R₁ by thermal transition (Publication [3]).

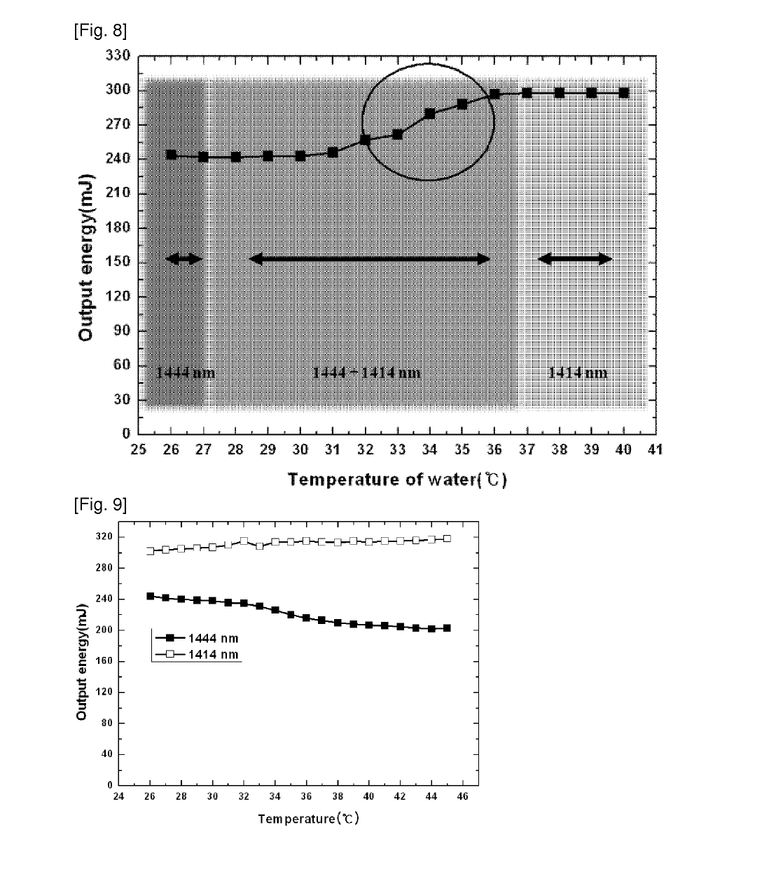

FIG. 8 shows experimental results that prove the above-described hypothesis. With regard to experimental conditions, the reflectivities of the output coupler was adjusted such that the other possible lasing wavelengths were not oscillated and only the 1414 nm and 1444 nm wavelengths were obtained, the reflectances for 1414 nm and 1444 nm wavelengths were set to the same value of 85%, pumping energy was fixed at 44 J, only the temperature of cooling water was increased, and then the change in output wavelength was observed.

As can be seen from FIG. 8, as increased the temperature of cooling water, the output wavelength shifts from 1444 nm to 1414 nm and, at the same time, the output energy was increased.

FIG. 9 shows the differences in output energy between two wavelengths as increased the temperature of cooling water using the two output couplers, the reflectivities of which have been adjusted to obtain only the 1444 nm and 1414 nm wavelengths with an input energy of 44 J.

As can be seen from the results of FIG. 9, the 1414 nm wavelength shows minimal variation in output energy variation as increased the temperature of cooling water, while the 1444 nm wavelength shows high variation in output energy. Furthermore, it can be seen that the output energy of the 1444 nm wavelength is lower than that of the 1414 nm wavelength.

Accordingly, it can be seen that in order to obtain a laser beam using an Nd:YAG crystal at a spectrum region of 1400 nm, the 1414 nm wavelength is superior in the stability of output energy than 1444 nm.

According to the results of calculation using Equation 1, in order to obtain the 1414 nm wavelength, the reflectivity at the 1414 nm wavelength should be at least 5% higher than that for the 1444 nm reflectivity. That is, when the reflectivity for the 1414 nm wavelength at the output coupler is 93%, the reflectivity at the 1444 nm wavelength should be less than 88%.

The actual Nd:YAG laser apparatus for oscillating the 1414 nm wavelength will now be described.

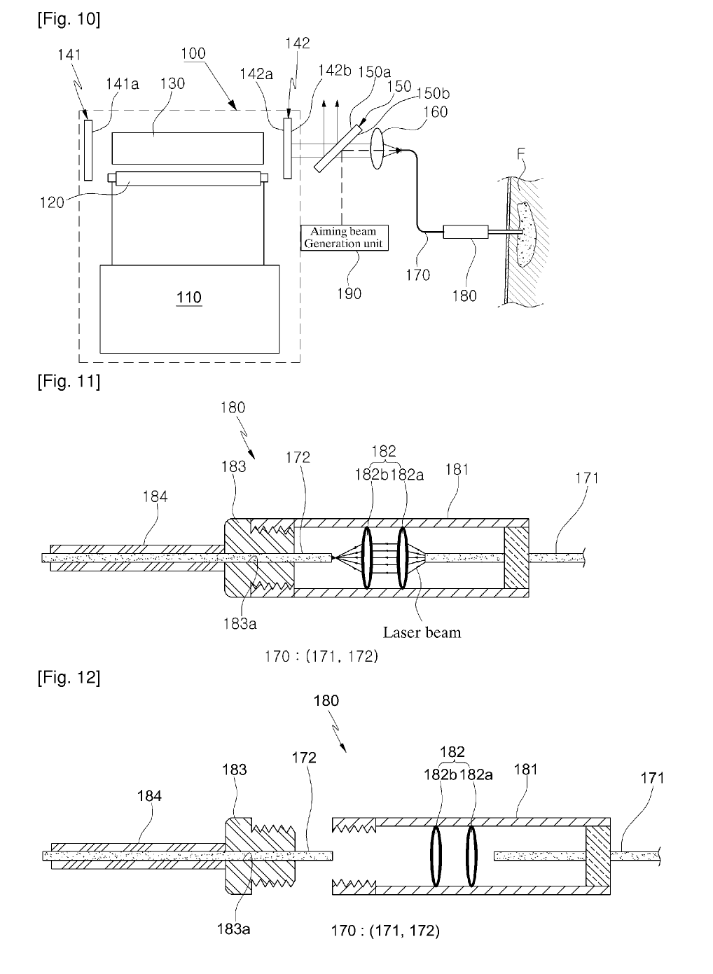

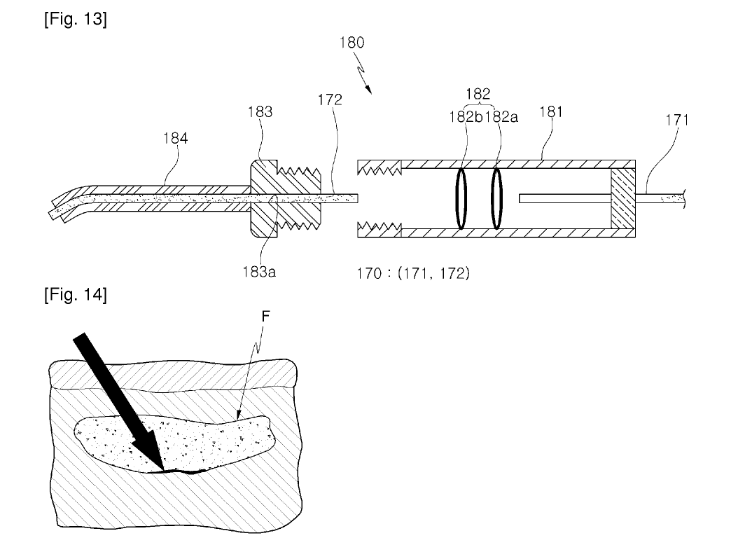

FIG. 10 is a diagram showing the construction of the fat removal 1414 nm Nd:YAG laser apparatus for direct irradiation into fat according to the present invention.

As shown in FIG. 10, the fat removal 1414 nm Nd:YAG laser apparatus for direct irradiation into fat according to the present invention includes an Nd:YAG laser body 100, a beam combiner and filter 150, a convergence lens 160, an optical fiber 170, and a cannula 180.

The Nd:YAG laser body 100 includes a flashlamp 120 which is supplied electrical power from a power supply unit 110 and emit light, an Nd:YAG rod 130 which radiate the laser wavelength after absorbing the pumping light from the flashlamp 120, and a high reflector 141 and output coupler 142 respectively located on two sides of the Nd:YAG rod 130 and reflect laser wavelength radiates from the Nd:YAG rod 130.

The convergence lens 160 converge a laser beam output through the output coupler 142 by condensing the laser beam, and may be formed of, for example, a convex lens.

The laser beam converged into optical fiber 170 by the convergence lens 160 and delivers the laser beam into fat F.

The cannula 180 is coupled to the output end of the optical fiber 170, and enables the optical fiber 170 to extend through a hypodermic region without being bent, thus the laser beam is delivered by the optical fiber 170, into hypodermic fat.

The construction of the cannula 180 and the optical fiber 170 will be described in greater detail below.

Since the laser beam is directly irradiated into the fat F through the optical fiber 170 and the cannula 180 as described above, it is not necessary to consider the loss of the laser beam resulting from absorption by water, unlike in the case where a laser beam is irradiated from outside the skin, so that it is possible to use the 1414 nm wavelength which has a higher absorption coefficient for fat.

Furthermore, with regard to the laser beam output through the output coupler 160, both end surfaces of the Nd:YAG rod 130, the internal surface 141a of the high reflector 141 and the internal surface 142a and external surface 142b of the output coupler 142 are coated to obtain only a laser beam with a wavelength of 1414 nm.

The coating specifications for the oscillation only the 1414 nm laser beam will now be described in greater detail.

In order to obtain only the 1414 nm laser beam, an embodiment of the present invention is configured such that the two surfaces of the Nd:YAG rod 130 are anti-reflectively coated in order to prevent reflection a laser beam in the 1050˜1450 nm region; the internal surface 141a of the high reflector 141 is coated to totally reflect a laser beam with a wavelength of 1414 nm.

The internal surface 142a of the output coupler 142 is coated to have a reflectivity less than 48% in the 1050˜1150 nm wavelength region (specially, it is preferable to have a reflectivity less than 25% at a wavelength of 1064 nm), a reflectivity less than 79% in the 1300˜4360 nm region (specially, less than 58% in the 1319˜4339 nm wavelength), a reflectivity of 93% at a wavelength of 1414 nm, and a reflectivity less than 88% at a wavelength of 1444 nm.

As described above, when output coupler have the reflectivities of 93% and less than 88% at 1414 nm and 1444 nm, respectively, the single oscillation of 1414 nm can be obtained. Since a laser has a reflectivity optimized for the operating condition thereof, it is apparent that the reflectivity for the 1414 nm wavelength is not limited to 93%. That is, depending on the operating condition of the laser, a lower reflectivity is appropriate for high input energy and higher reflectivity is appropriate for low input energy.

Furthermore, since three types of various methods may exist to generate only the 1414 nm wavelength as presented in methods 1, 2, 3 and 4, it is apparent that the reflectivities for various possible lasing wavelengths are not limited.

Furthermore, in the embodiment of the present invention, the beam combiner and filter 150 is provided between the output coupler 142 and the convergence lens 160. The incident surface 150a of the beam combiner and filter 150 is coated to have a transmitivity more than 98.5% for the 1414 nm wavelength, and to have a reflectivity of 99% for the 1064 nm wavelength in order to prevent for the case where Amplified Spontaneous Emission (ASE) with a wavelength of 1064 nm which have the highest stimulated emission cross section in the Nd:YAG rod 130 or the case where the resonator mirror or Nd:YAG crystal is contaminated, so that the reflectivity varies, with the result that the 1064 nm wavelength may be have the chance of the oscillation.

In practice, as apparently described in Publication [1], although the loss of the 1064 nm wavelength in the resonator is considerably high, there is a possibility of the gain of the 1414 nm wavelength being not achieved around the Nd:YAG rod due to the thermal lens effect of Nd:YAG depend on input energy, so that it is essential to use the beam combiner and filter 150.

Furthermore, the fat removal 1414 nm Nd:YAG laser apparatus according to the embodiment of the present invention further includes an aiming beam generation unit 190 for radiation of visible light, to the exit surface 150b of the beam combiner and filter 150, an aiming beam for indicating the location into which a laser beam with a wavelength of 1414 nm that is obtained by the Nd:YAG rod 130 is irradiated inside the skin so as to remove fat.

It is preferred that the wavelength of the aiming beam radiation from the aiming beam generation unit 190 correspond to a low output power, for example, in the red wavelength region of 630˜660 nm.

Since the location of a laser beam with a wavelength of 1414 nm irradiated into the skin (more specifically, the location of the cannula 180 inside the skin) can be found by emitting a low output power of aiming beam in the red wavelength region of 630˜660 nm, there is an advantage of maximizing the convenience of application and efficiency.

In the embodiment of the present invention, the exit surface 150b of the beam combiner and filter 150 is coated to have a reflectivity more than 90% for the 630˜660 nm wavelength and a transmissivity equal to or greater than 99% for the 1414 nm wavelength, and the convergence lens 160 is anti-reflectively coated to reflect laser beams with wavelengths of 1414 nm and 630˜660 nm.

Furthermore, in the fat removal 1414 nm Nd:YAG laser apparatus for direct irradiation into fat according the embodiment of the present invention, the optical fiber 170 is formed of a fixed optical fiber 171 configured to deliver a laser beam converged by the convergence lens 160 and a disposable optical fiber 172 inserted into the human body and configured to irradiate the laser beam which delivered by the fixed optical fiber 171 into the fat.

Furthermore, in the fat removal 1414 nm Nd:YAG laser apparatus for direct irradiation into fat according to the embodiment of the present invention, the cannula 180 includes a cannula body 181 provided such that the output end of the fixed optical fiber 170 is fixed to one side thereof and the input end of the disposable optical fiber 172 is detachably provided on the other side thereof and configured to be gripped by a user, light transmission means 182 provided between the output end of the optical fiber 170 and the input end of the disposable optical fiber 172 inside the cannula body 181 and configured to deliver a laser beam so that the laser beam output from the output end of the fixed optical fiber 171 smoothly enters the input end of the disposable optical fiber 172, and a cannula tip 184 fitted over the disposable optical fiber 172 so that the disposable optical fiber 172 can be smoothly inserted into the human body and extend underneath skin without being bent.

Furthermore, the fat removal 1414 nm Nd:YAG laser apparatus for direct irradiation into fat according to the embodiment of the present invention further includes a disposable optical fiber holder 183 which is detachably fitted into the rear end of the cannula body 181 and through which a through hole 183a through which the disposable optical fiber 172 is inserted and passes is formed in the direction of the propagation of a laser beam.

Above the laser beam coupling system 182 includes a collimate lens 182a provided inside the cannula body 181 so that it is located in front of the output end of the fixed optical fiber 171 in the direction of the propagation of a laser beam and configured to make the converge beam by emitted from the fixed optical fiber 171 into parallel beam, and a focusing lens 182b provided inside the cannula body 181 so that it is located in front of the collimate lens 182a and configured to focus a parallel laser beam propagating through the collimate lens 182a and cause the parallel laser beam converge to enter the input end of the disposable optical fiber 172.

Here, the focal length of the collimate lens 182a and the focusing lens 182b should be change depend on the Numerical Aperture (N.A.) of the optical fibers 171 and 172 and the diameter of parallel beam, in which case a laser beam is focused in front of the incident surface of the disposable optical fiber 172, as shown in FIG. 11, so that damage to the disposable optical fiber 172 can be prevented.

According to the above-described construction, the optical fiber 170 for delivery a laser beam is separated into the fixed optical fiber 171 and the disposable optical fiber 172 actually inserted into a human body and configured to irradiate a laser beam into the human body. Accordingly, since it is necessary to dispose of only the disposable optical fiber 172 inserted into the human body, there is an advantage of reducing the cost burden resulting from the disposal of an optical fiber in the case where the optical fiber is disposed of and there is another advantage of improving the convenience of use by eliminating sterilize of the optical fiber whenever treatment using the laser apparatus is performed.

In the fat removal 1414 nm Nd:YAG laser apparatus according the embodiment of the present invention, the output of a laser beam obtained by the Nd:YAG laser has a repetition rate in the range of 1˜100 Hz, a energy per pulse in the range of 10˜10,000 mJ, optical output power in the range of 0.5˜50 W, and a pulse width in the range of 10 μs˜1000 ms.

Moreover, when the 1414 nm laser beam is used, as in the present invention, there may occur a case where the 1414 nm laser beam is not entirely absorbed by the fat F but is propagated to adjacent tissues, in which case there is an advantage in that damage to the adjacent tissues is much less than in the case where a laser beam with other wavelength such as 1064 nm and 1319 nm are used because the absorption coefficient of the 1414 nm wavelength for water is very higher than 1064 nm and 1319 nm wavelength.

The operation of the fat removal 1414 nm Nd:YAG laser apparatus for direct irradiation into fat according to the present invention will now be described.

FIG. 14 is a conceptual diagram showing the case where a 1414 nm wavelength laser beam is used according to the present invention.

First, since the two surfaces of the Nd:YAG rod 130, the internal surface 141a of the high reflector 141, and the internal surface 142a and external surface 142b of the output coupler 142 are coated according to the above-described coating specifications, only a 1414 nm laser beam is radiation from the output coupler 142 when electrical power is supplied to flashlamp 120.

In this case, a 1064 nm laser beam that can be oscillated at high electrical input energy is filtered by the beam combiner and filter 150, so that it cannot enter the optical fiber 170.

Meanwhile, the 1414 nm laser beam propagating through the optical combiner and filter 150 is converged by the convergence lens 160, is delivered by the optical fiber 170 and cannula 180, is directly irradiated into the hypodermic fat F, and then dissolves fat.

Furthermore, as shown in FIG. 14, since the 1414 nm wavelength used in the present invention has high absorption coefficients for both fat and water, the water of the human body prevents the heat transfer by absorbing a laser when the user erroneously irradiates the laser into tissues adjacent to fat. Accordingly, when a user erroneously irradiates a laser into tissues adjacent to fat, damage to the adjacent tissues (the portion illustrated in black in FIG. 14) can be minimized.

Meanwhile, an aiming beam radiated by the aiming beam generation unit 190 is reflected from the beam combiner and filter 150, propagates through the convergence lens 160, and incident the optical fiber 170, thereby indicating the location of end of the cannula 180.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A fat removal 1414 nm Nd:YAG laser apparatus for direct radiation into fat, comprising:
a flashlamp for receiving power from a power supply unit and emitting pumping light;
an Nd:YAG rod for radiating a laser wavelength and amplifying the laser wavelength by absorbing the pumping light.
a high reflector and an output coupler located on two sides of the Nd:YAG rod to reflect and transmit the laser wavelength radiated from the Nd:YAG rod;
a convergence lens for converging the laser beam output from the output coupler;
an optical fiber for guiding the laser beam converged by the convergence lens; and
a cannula coupled to an output end of the optical fiber and configured to allow the optical fiber to extend underneath skin without being bent, thereby guiding the laser beam delivered by the optical fiber to hypodermic fat;
wherein both end surfaces of the Nd:YAG rod, an internal surface of the high reflector, and internal and external surfaces of the output coupler are coated such that only a laser beam with a wavelength of 1414 nm is radiated through the output coupler.

2. The fat removal 1414 nm Nd:YAG laser apparatus as set forth in claim 1, wherein:
in order to obtain only the laser beam with a wavelength of 1414 nm, the two surfaces of the Nd:YAG rod are anti-reflectively coated not to reflect a laser beam with a wavelength in a range of 1050˜4450 nm;
the internal surface of the high reflector is coated to totally reflect the laser beam with a wavelength of 1414 nm, to have a reflectivity equal to or less than that at 1414 nm, to have a reflectivity of 30% less than that at 1414 nm in a wavelength band of 1050˜4150 nm and to have a reflectivity of 20% less than that at 1414 nm in a wavelength band of 1300˜4360 nm;
the internal surface of the output coupler is coated to have a reflectivity less than 48% in a wavelength band of 1050˜4150 nm, particularly, a reflectivity less than 26% at a wavelength of 1064 nm, and to have a reflectivity less than 79% in a wavelength band of 1300˜4360 nm, particularly, a reflectivity less than 58% at a wavelength band of 1319˜4339 nm, and to cause a reflectivity at wavelength of 1414 nm to be 5% or more higher than that for a wavelength of 1444 nm;
the external surface of the output coupler is anti-reflectively coated in a wavelength band of 1050˜4450 nm; and
the convergence lens is anti-reflectively coated not to reflect at a wavelength of 1414 nm.

3. The fat removal 1414 nm Nd:YAG laser apparatus as set forth in claim 2, wherein the internal surface of the output coupler is coated to have a reflectivity of 93% at a wavelength of 1414 nm, and to have a reflectivity less than 88% at a wavelength of 1444 nm.

4. The fat removal 1414 nm Nd:YAG laser apparatus as set forth in claim 2, wherein:
a beam combiner and filter is provided between the output coupler and the convergence lens; and
an incident surface of the beam combiner and filter is coated to have a transmitivity equal to or greater than 98.5% at a wavelength of 1414 nm and to have a reflectivity of 99.5% at a wavelength of 1064 nm.

5. The fat removal 1414 nm Nd:YAG laser apparatus as set forth in claim 4, further comprising an aiming beam generation unit for outputting, to an exit surface of the beam combiner and filter, an aiming beam for indicating a location at which the laser beam with a wavelength of 1414 nm radiated by the Nd:YAG rod so as to remove fat is radiated inside skin.

6. The fat removal 1414 nm Nd:YAG laser apparatus as set forth in claim 5, wherein:
the aiming beam output from the aiming beam generation unit has a wavelength of 633 nm;
an incident surface of the beam combiner and filter is coated to have a transmitivity equal to or greater than 98.5% at a wavelength of 1414 nm and to have a reflectivity equal to or greater than 99.5% at a wavelength of 1064 nm;
the exit surface of the beam combiner and filter is coated to have a reflectivity equal to or greater than 90% at a wavelength of 633 nm and to have a transmitivity equal to or greater than 99.5% at a wavelength of 1414 nm; and
the convergence lens is anti-reflectively coated not to reflect at wavelengths of 1414 nm and 633 nm.

7. The fat removal 1414 nm Nd:YAG laser apparatus as set forth in claim 1, wherein:
the optical fiber is separated into a fixed optical fiber configured to deliver the laser beam converged by the convergence lens and a disposable optical fiber inserted into a human body and configured to irradiate the laser beam delivered by the fixed optical fiber into the human body; and
the cannula comprises:
a cannula body configured such that an output end of the fixed optical fiber is fixed onto one side thereof and an input end of the disposable optical fiber is detachably provided on a remaining side thereof, and configured to be gripped by a user;
laser beam coupling system means provided between an output end of the optical fiber and an input end of the disposable optical fiber inside the cannula body, and configured to guide the laser beam so that the laser beam radiated from an output end of the fixed optical fiber can enter an input end of the disposable optical fiber without hindrance;
a straight cannula tip fitted over the disposable optical fiber, and configured to allow the disposable optical fiber to be inserted into the human body without being bent underneath skin; and
a curved cannula tip fitted over the disposable curved optical fiber, and configured to allow the disposable curved optical fiber to be inserted into the human body the curved cannula tip being utilized for a curved surface of the human body, such as a face, and being very helpful in face lifting.

8. The fat removal 1414 nm Nd:YAG laser apparatus as set forth in claim 7, further comprising a disposable optical fiber holder detachably fitted into a rear end of the cannula body, and configured such that a through hole through which the disposable optical fiber is inserted and passes is formed therethrough in a direction of propagation of the laser beam.

9. The fat removal-dedicated 1414 nm Nd:YAG laser apparatus as set forth in claim 7, wherein the light transmission means comprises:
a collimate lens provided inside the cannula body so that it is located in front of the output end of the fixed optical fiber in a direction of propagation of the laser light, and configured to increase a size of the laser beam by converting the laser beam exiting from the fixed optical fiber into parallel light; and
a focusing lens provided inside the cannula body so that it is located in front of the collimate lens, and configured to focus the parallel laser beam passing through the collimate lens and cause the parallel laser beam to enter the input end of the disposable optical fiber.

10. The fat removal-dedicated 1414 nm Nd:YAG laser apparatus as set forth in claim 1, wherein an output of the laser beam generated by the Nd:YAG rod has a repetition rate in a range of 1˜100 Hz, an energy per pulse in a range of 1˜10,000 mJ, power in a range of 0.5˜100 W, and a pulse width in a range of 1 μs˜1000 ms.
